(19) United States
(12) Patent Application Publication
(10) Pub. No.: US 2011/0213150 A1
(43) Pub. Date: Sep. 1, 2011

(54) PREPARATION OF CRYSTALLINE PALONOSETRON HYDROCHLORIDE

(75) Inventors: Srinivas Katkam, Secunderabad (IN); Rajeshwar Reddy Sagyam, Hyderabad (IN); Vishnuvardhan Sunkara, Hyderabad (IN); Sridhar Munagala, Hyderabad (IN); Murali Mohan Muttavarapu, Krishna (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/103,277

(22) Filed: May 9, 2011

Related U.S. Application Data
(63) Continuation of application No. PCT/US2009/063846, filed on Nov. 10, 2009.
(60) Provisional application No. 61/142,514, filed on Jan. 5, 2009.

(30) Foreign Application Priority Data
Nov. 11, 2008 (IN) 2769/CHE/2008

(51) Int. Cl. C07D 453/02

(52) U.S. Cl. 546/99

(57) ABSTRACT

Processes for the preparation of palonosetron hydrochloride and its crystalline forms.

Aspects of the present application relate to palonosetron and processes for the preparation of crystalline forms of palonosetron hydrochloride.

The drug compound having the adopted name “palonosetron hydrochloride” has a chemical name (3aS)-2-[(S)-1-azabicyclo[2.2.2]oct-3-yl]-2,3,3a,4,5,6-hexahydro-1-oxo-1H-benz[de]isoquinoline hydrochloride, and is represented by structural formula I.

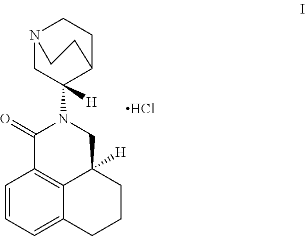

Palonosetron hydrochloride is an antiemetic and antinauseant agent, and is sold using the trademark ALOXI® in the form of injectables and capsules.

U.S. Pat. No. 5,202,333 discloses palonosetron, its pharmaceutically acceptable salts, and processes for their preparation. Further, it discloses the use of ethanol for crystallization of palonosetron hydrochloride. The product obtained is characterized by a melting point of 296-297° C.

C. Chan et al., “Inhibitors of cholesterol biosynthesis. 1. 3,5-Dihydroxy-7-(N-imidazolyl)-6-heptenoates and -heptanoates, a novel series of 3-hydroxy-3-methylglutarate-CoA reductase inhibitors,” Journal of Medicinal Chemistry, 1993, 36, (23), pp 3646-3657, discloses the crystallization of palonosetron hydrochloride from ethanol. The product obtained is characterized by X-ray crystallographic data. The diffraction photographs show monoclinic symmetry. The lattice constants are a=8.996 Å, b=7.555 Å, c=12.624 Å, and β=98.080°.

U.S. Pat. No. 5,567,818 discloses a process for the crystallization of palonosetron hydrochloride from isopropanol and water. The process involves dissolving a diastereomeric mixture of 97% 3aS and 3% 3aR palonosetron hydrochloride in isopropanol. The solution is heated to reflux, then water and additional isopropanol are added. The mixture is distilled, cooled over 2 hours to 20° C., then cooled to 5° C., and stirred for approximately 18 hours to give a crystalline precipitate. The precipitate is isolated by filtration, then dried in nitrogen vacuum oven at 68° C. to give 99.1% pure palonosetron hydrochloride with a melting point of 303° C.

U.S. Pat. No. 5,510,486 discloses a process for the crystallization of palonosetron hydrochloride from isopropanol and water. The disclosed process involves dissolving palonosetron hydrochloride in isopropanol and water at reflux temperature, followed by the addition of a second lot of isopropanol. The mixture is distilled, allowed to cool to room temperature, and further cooled in an ice-water bath. The isolated crystalline palonosetron hydrochloride has a melting point of 303° C.

U.S. Patent Application Publication No. 2008/0058367 A1 discloses a crystalline form of palonosetron hydrochloride, characterized by an X-ray powder diffraction pattern with principal peaks approximately at 7.1, 13.8, 14.2, 15.8, 18.5, 19.7, 20.0 and 24.4±0.2 degrees 2-theta, which is obtained by repeated crystallizations from methanol.

K. Ravikumar et al., “An orthorhombic polymorph of palonosetron hydrochloride,” Acta Crystallographica (2007), E63, o1404-o1406, discloses an orthorhombic polymorphic crystalline form of palonosetron hydrochloride obtained by crystallization from dimethylformamide with lattice constants of a=7.497 Å, b=9.029 Å, and c=25.045 Å.

International Application Publication No. WO 2008/051564 A2 discloses two crystalline forms of palonosetron hydrochloride. The first form is characterized by powder X-ray diffraction with peaks at about 13.0, 15.4, and 17.5 degrees two-theta. The first form is obtained by crystallization of a diastereomeric mixture of palonosetron hydrochloride from methanol, isopropanol, water, or mixtures thereof, evaporating the solvent until dry, and drying the solid under vacuum at 70° C. The second form is characterized by powder X-ray diffraction with peaks at about 12.1, 15.4, and 17.5 degrees two-theta. The second form is obtained by crystallization of a diastereomeric mixture of palonosetron hydrochloride from isopropanol and water (95:5 mixture) or methanol, isopropanol, and water, or by storing the first polymorphic form at 100% relative humidity for 1 week.

International Application Publication No. WO 2008/073757 A1 discloses two pure crystalline forms of palonosetron hydrochloride, designated Form I and Form II, and amorphous palonosetron hydrochloride. Form I is prepared by crystallization from an ethanol solution of palonosetron hydrochloride held at ambient temperature for one week. Form II is prepared by crystallization from a hot ethanol solution of palonosetron hydrochloride. The crystals are filtered immediately upon cooling to room temperature and dried. The amorphous form is prepared by lyophilization of a solution of the compound in water.

Despite the existence of different processes for preparing crystalline forms of palonosetron hydrochloride, there exists an ongoing need for convenient and consistent processes for preparing polymorphic forms of palonosetron hydrochloride, which processes are amenable to large-scale production.

### SUMMARY

Aspects of the present invention include processes for preparing palonosetron hydrochloride and its crystalline polymorphic forms.

For example, there are provided processes for the preparation of preparation of palonosetron hydrochloride of formula I, embodiments comprising:

(a) hydrogenating a compound of formula IV,

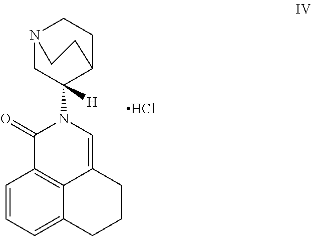

in the presence of a hydrogenation catalyst and n-propanol;

(b) providing a suspension of the step a) product in methanol;

(c) maintaining the suspension at a temperature of about 45° C. to the reflux temperature; and

(d) isolating palonosetron hydrochloride of formula I.

For example, there are provided processes for the preparation of a crystalline form of palonosetron hydrochloride characterized by an X-ray powder diffraction pattern with principal peaks approximately at 7.1, 13.8, 14.2, 15.8, 18.5, 19.7, 20.0, and 24.4, ±0.2 degrees 2-theta (hereinafter referred as “Form A”), embodiments comprising:

(a) providing a suspension of palonosetron hydrochloride n-propyl alcohol or a nitrile solvent;

(b) maintaining the suspension at a temperature of about 50° C. to a temperature up to the boiling point of the solvent; and

(c) isolating crystalline Form A.

Also for example, there are provided processes for the preparation of a crystalline form of palonosetron hydrochloride characterized by an X-ray powder diffraction pattern with principal peaks approximately at 9.8, 11.3, 12.9, 15.3, 16.1, 16.3, 17.5, 22.0, and 25.0±0.2 degrees 2-theta (hereinafter referred as “Form B”), embodiments comprising:

(a) providing a suspension of palonosetron hydrochloride in a ketone solvent, optionally in combination with an alcohol;

(b) maintaining the suspension at a suitable temperature; and

(c) isolating crystalline Form B.

For another example, there are provided processes for the preparation of a mixture of palonosetron hydrochloride crystalline Forms A and B, embodiments comprising:

(a) providing a suspension or solution of palonosetron hydrochloride in n-propyl alcohol or a nitrile solvent, optionally in combination with a second alcohol or ketone solvent, or mixtures thereof;

(b) maintaining the mixture at temperatures in the range of about 0° C. to about 40° C.; and

(c) isolating a mixture of crystalline forms.

### BRIEF DESCRIPTION OF THE DRAWINGS

FIG. 1 illustrates an X-ray powder diffraction (XRPD) pattern of palonosetron hydrochloride Form A, as prepared in Example 5.

FIG. 2 illustrates an XRPD pattern of palonosetron hydrochloride Form B, as prepared in Example 1.

FIG. 3 illustrates an XRPD pattern of a mixture of crystalline Forms A and B of palonosetron hydrochloride, as prepared in Example 7.

FIG. 4 illustrates an XRPD pattern of palonosetron hydrochloride Form A as prepared in Example 9.

FIG. 5 illustrates an XRPD pattern of palonosetron hydrochloride Form A as prepared in Example 10.

FIG. 6 illustrates a differential scanning calorimetry (DSC) curve of palonosetron hydrochloride Form A as prepared in Example 10.

FIG. 7 illustrates a thermogravimetric analysis (TGA) curve of palonosetron hydrochloride Form A as prepared in Example 10.

FIG. 8 is a schematic representation of a process for preparing palonosetron hydrochloride.

### DETAILED DESCRIPTION

Aspects of the present invention include processes for preparing crystalline forms of palonosetron hydrochloride and mixtures thereof.

For example, there are provided processes for the preparation of crystalline palonosetron hydrochloride of formula I, embodiments comprising:

a) hydrogenating a compound of formula IV,

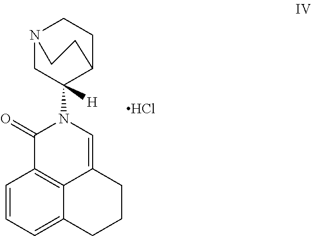

in the presence of a hydrogenation catalyst and n-propanol;

b) providing a suspension of the step a) product in methanol;

c) maintaining the suspension at temperatures about 45° C. to the reflux temperature; and

d) isolating palonosetron hydrochloride of formula I.

Step a) involves hydrogenating a compound of the formula IV in the presence of a hydrogenation catalyst and n-propanol.

The process of step a) for hydrogenation may be carried out using a hydrogenation catalyst such as palladium on carbon (Pd/C), palladium on barium sulfate (Pd/BaSO₄), PtO₂, and the like. For example, 10% Pd/C having type 487 or 489, or equivalent grades, can be used as the hydrogenation catalyst.

In an embodiment, about 30% to about 60%, or about 40% to about 60%, by weight of wet 10% Pd/C is used as the hydrogenation catalyst. These percentages are based on the amount of the compound of formula IV.

The hydrogenation may be performed at hydrogen gas pressures about 8-12 Kg/cm² and at temperatures about 25° C. to about the reflux temperature of n-propanol. For example, the reduction may be carried out at temperatures about 55° C. to about 60° C.

After completion of the reaction, the mixture may be filtered to remove the catalyst under hot or cooled conditions, the catalyst is washed with n-propanol, and palonosetron HCl may be isolated using techniques known in the art.

In an embodiment, palonosetron hydrochloride may be isolated by concentrating the reaction mass or by distilling the reaction mass to a minimum volume, followed by cooling to temperatures less than about 35° C. and separating the formed compound.

Step b) involves providing a suspension of the step a) product in methanol.

A suspension of step a) product in methanol may be provided by combining the product with methanol, optionally under a nitrogen atmosphere, or by dissolving the product obtained in step a) in methanol, followed by concentration to the desired extent to produce a suspension.

The suspension of palonosetron hydrochloride may be provided at temperatures ranging from about 20° C. up to the boiling point of the methanol.

Step c) involves maintaining the suspension at temperatures about 45° C. to the reflux temperature.

The suspension of step b) is maintained at temperatures about 45° C. to the reflux temperature of methanol for a suitable time period, such as about 10 minutes to about 4 hours, or longer.

Step d) involves isolating palonosetron hydrochloride of formula I.

The suspension of step c) may be cooled to temperatures below about 35° C., such as temperatures about 0-5° C., and maintained for about 30 minutes to about 4 hours, or longer, and the product may be isolated using techniques known in the art. For example, it may be isolated using filtration by gravity or by suction, centrifugation, decantation, and the like.

Optionally, steps b) to d) of the above process may be repeated one or more times, to obtain palonosetron hydrochloride of a desired purity.

In a particular embodiment, there is provided a process for the preparation of palonosetron hydrochloride, comprising:

(a) reacting a compound of formula IV with hydrogen, in the presence of Pd/C and n-propanol;

(b) providing a suspension of the step a) product in methanol;

(c) maintaining the suspension at temperatures about 45° C. to the reflux temperature; and

(d) isolating palonosetron hydrochloride.

In an aspect, there are provided processes for the preparation of crystalline Form A of palonosetron hydrochloride, embodiments comprising:

(a) providing a suspension of palonosetron hydrochloride in n-propyl alcohol or a nitrile solvent;

(b) maintaining the suspension at temperatures about 50° C. to the boiling point of the solvent; and

(c) isolating crystalline Form A.

Step (a) involves providing a suspension of palonosetron hydrochloride in n-propyl alcohol or a nitrile solvent.

A suspension of palonosetron hydrochloride in methanol or n-propyl alcohol or a nitrile solvent may be provided from the chemical reaction by which the compound is prepared, or by combining isolated palonosetron hydrochloride with n-propyl alcohol or a nitrile solvent, optionally under a nitrogen atmosphere. Any form of palonosetron hydrochloride, such as amorphous, crystalline, or mixtures thereof, in any proportions, obtained by any method, may be used for providing the suspension.

Nitrile solvents may comprise acetonitrile and/or propionitrile. In a specific embodiment, acetonitrile is used for providing the suspension of palonosetron hydrochloride.

The suspension of palonosetron hydrochloride may be provided at temperatures ranging from about 20° C. up to the boiling point of the solvent.

The suspension of palonosetron hydrochloride may also be provided by dissolving palonosetron hydrochloride in a desired solvent, followed by concentration to a desired extent to produce a suspension.

Step (b) involves maintaining the suspension at temperatures about 50° C. to the boiling point of the solvent.

The suspension of (a) is maintained at temperatures about 50° C. or higher, for a suitable time period to facilitate the formation of a desired crystalline form.

The suspension of palonosetron hydrochloride may be maintained at a temperature of about 50° C. to a temperature up to the boiling point of the solvent used.

For example, the suspension of palonosetron hydrochloride may be maintained at the reflux temperature of the solvent used.

The obtained suspension may be maintained at the selected temperature for about 30 minutes to about 10 hours, or longer, to facilitate the conversion of other polymorphic forms of palonosetron hydrochloride to the desired crystalline form. For example, the suspension of palonosetron hydrochloride is maintained for about 1 to 4 hours at the selected temperature.

Step (c) involves isolating the crystalline form.

The crystalline form may be isolated by the techniques known in the art. For example, it may be isolated by using filtration by gravity or by suction, centrifugation, decantation, and the like. For example, the crystalline form may be isolated by filtering the hot suspension obtained in (b).

After isolation, the solid may optionally be washed. A wet solid obtained from (c) may be dried in a tray dryer, vacuum oven, air oven, fluidized bed dryer, spin flash dryer, flash dryer, and the like. The drying may be carried out at temperatures about 45° C. to about 85° C., such as, for example, about 70° C., optionally under reduced pressure. The drying may be carried out for any time periods, such as, for example, for about 1 to about 25 hours, or longer, to give the desired crystalline form of palonosetron hydrochloride.

The steps (b-d) of the above process may be repeated more than one time to improve formation of crystalline form A of palonosetron hydrochloride and its chemical purity.

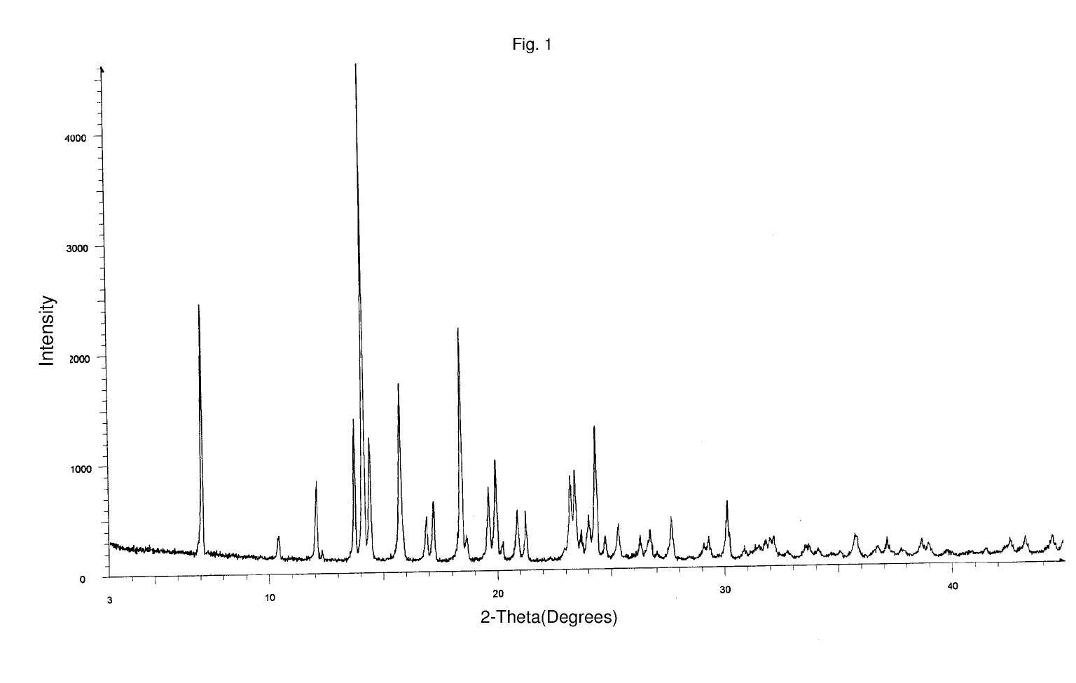

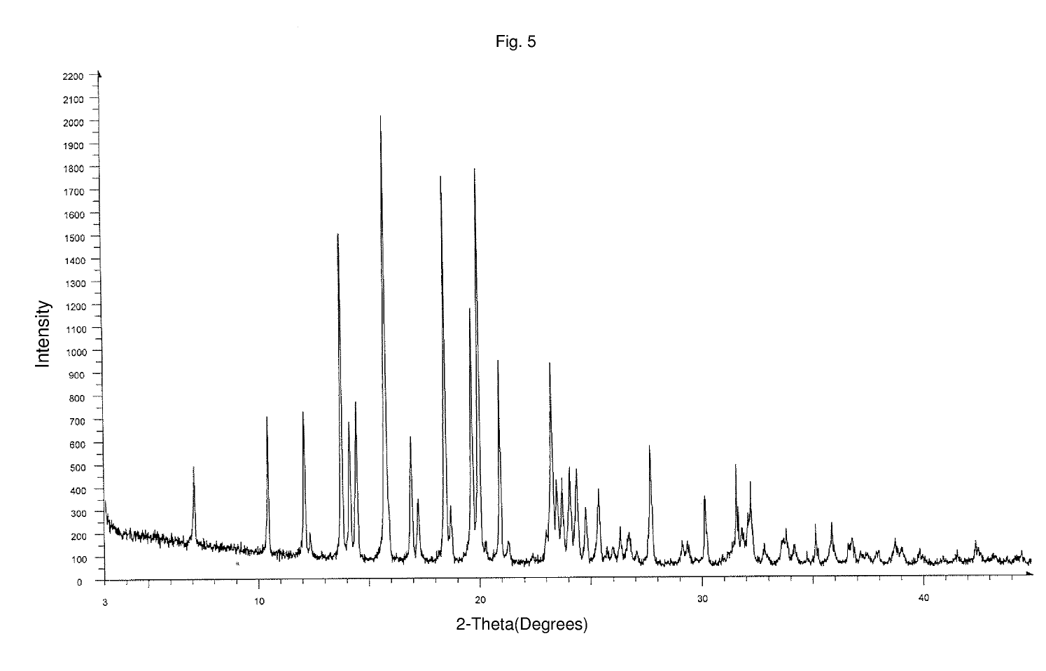

Form A has an XRPD pattern substantially in accordance with FIG. 1 or 5, and may have less than about 5%, or less than about 2%, or less than about 1%, by weight of other forms of palonosetron hydrochloride.

Form A obtained by a process of the present invention has a content of the 3aR isomer less than about 0.1%, as determined using HPLC.

For example, there is provided a process for the preparation of a crystalline Form B of palonosetron hydrochloride, which process comprises:

(a) providing a suspension of palonosetron hydrochloride in a ketone solvent, optionally in combination with an alcohol;

(b) maintaining the suspension at suitable temperature; and

(c) isolating the crystalline form.

Step (a) involves providing a suspension of palonosetron hydrochloride in a ketone solvent, optionally in combination with an alcohol.

A suspension of palonosetron hydrochloride in a ketone solvent, optionally in combination with an alcohol, may be provided from the chemical reaction by which it is prepared or by combining other polymorphic forms of palonosetron hydrochloride with the selected solvent.

The ketone solvent may comprise acetone, methyl ethyl ketone, and/or methyl isobutyl ketone. For example, acetone may be used for providing the suspension of palonosetron hydrochloride.

The alcohol that may be used in combination with the ketone may comprise a C₁-C₄ alcohol, such as, for example, methanol, ethanol, isopropanol, or mixtures thereof. For example, a combination of acetone and methanol may be used.

The suspension of palonosetron hydrochloride may be provided at a temperature from about 20° C. up to the boiling point of the solvent used.

Step (b) involves maintaining the suspension at a suitable temperature.

The suspension of Step (a) is maintained at a suitable temperature of about 40° C. up to the boiling point of the ketone solvent.

In specific embodiments, the suspension is maintained at the reflux temperature of the selected solvent.

The suspension obtained from (a) is maintained at the chosen temperature for a period of about 30 minutes to about 10 hours, or longer, to facilitate the conversion of other polymorphic forms of palonosetron hydrochloride to the desired crystalline form. For example, the suspension of palonosetron hydrochloride is maintained for 1 to 4 hours at the chosen temperature.

Step (c) involves isolating the crystalline form.

The crystalline form may be isolated by the techniques known in the art. For example, it may be isolated by filtration by gravity or by suction, centrifugation, decantation, and the like. For example, the crystalline form is isolated by filtering the hot suspension obtained in (b).

After isolation, the solid may optionally be washed. The wet solid obtained from (c) may be dried suitably in a tray dryer, vacuum oven, air oven, fluidized bed dryer, spin flash dryer, flash dryer, and the like. The drying may be carried out at temperatures about 45° C. to about 85° C., such as, for example, about 70° C., optionally under reduced pressure. The drying may be carried out for any time periods, such as, for example, about 1 to about 25 hours, or longer, to obtain the desired crystalline form of palonosetron hydrochloride.

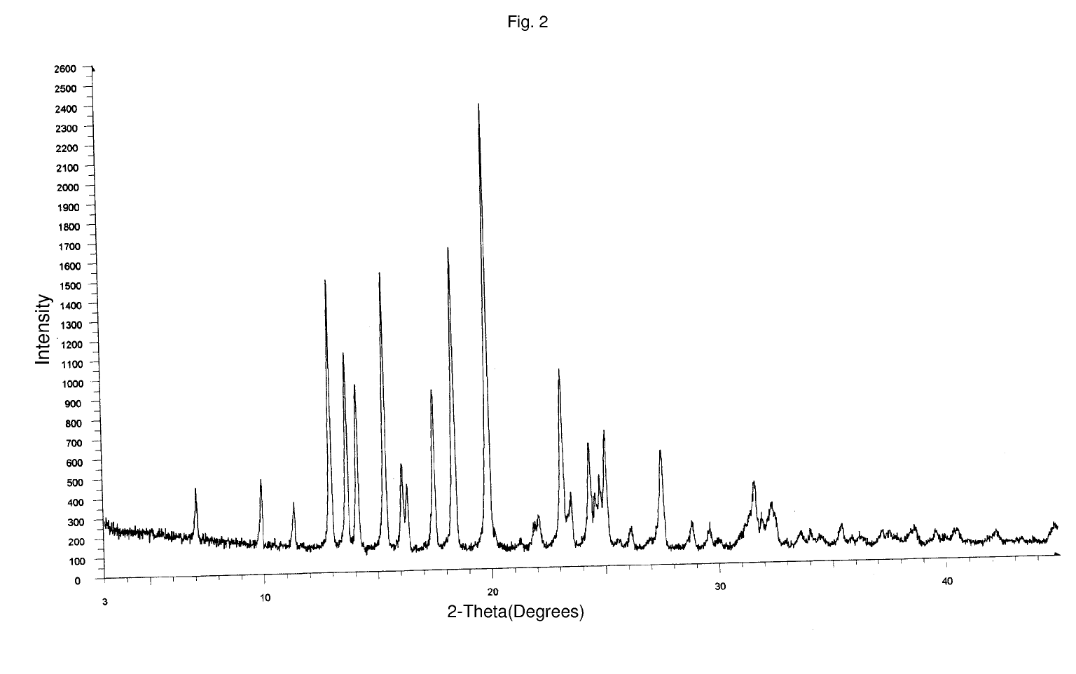

Form B has an XRPD pattern substantially in accordance with FIG. 2, and may have less than about 5%, or less than about 2%, or less than about 1% of other forms of palonosetron hydrochloride.

Form B obtained by the process of the present invention has a content of the 3aR isomer in the range of about 0.05% to about 15%, as determined using HPLC.

For example, there is provided a process for the preparation of a mixture of crystalline Forms A and B of palonosetron hydrochloride, which process comprises:

(a) providing a suspension or solution of palonosetron hydrochloride in n-propyl alcohol or a nitrile solvent, and optionally in combination with a second alcohol or a ketone solvent;

(b) maintaining the mixture at temperatures in the range of about 0° C. to about 40° C.; and

(c) isolating the mixture of crystalline forms.

Step (a) involves providing a suspension or solution of palonosetron hydrochloride in n-propyl alcohol or a nitrile solvent, and optionally in combination with a second alcohol or a ketone solvent.

A suspension of palonosetron hydrochloride in n-propyl alcohol or a nitrile solvent, and optionally in combination with a second alcohol or a ketone solvent, be provided from the chemical reaction by which the compound is prepared or by combining any polymorphic forms of palonosetron hydrochloride with the selected solvent.

The nitrile solvent may comprise acetonitrile and/or propionitrile. For example, acetonitrile is used for providing the suspension of palonosetron hydrochloride.

The second alcohol that may be used in combination with the ketone may comprise a C₁-C₄ alcohol, such as, for example, methanol, ethanol, isopropanol, and any mixtures thereof. For example, a combination of acetone and methanol may be used.

The ketone solvent may comprise acetone, methyl ethyl ketone, and/or methyl isobutyl ketone. For example, acetone may be used for providing the suspension of palonosetron hydrochloride.

The suspension of palonosetron hydrochloride may be provided at temperatures ranging from about 20° C. up to the boiling point of the solvent used.

Step (b) involves maintaining the suspension at temperatures in the range of about 0° C. to about 40° C.

The suspension of (a) is maintained at temperatures less than about 40° C. and for a suitable time period for facilitating the conversion of other polymorphic forms of palonosetron hydrochloride to the desired mixture of crystalline form A and B.

The suspension of palonosetron hydrochloride is maintained at a temperature of below 40° C. and can be as low as about 0° C. For example, the suspension of palonosetron hydrochloride is maintained at a temperature of about 25° C. to 35° C.

The obtained suspension is maintained at the chosen temperature for about 30 minutes to about 10 hours, or longer, to facilitate the conversion of other polymorphic forms of palonosetron hydrochloride to the desired crystalline form. For example, the suspension of palonosetron hydrochloride is maintained for about 1 to about 4 hours at the chosen temperature.

Step (c) involves isolating the crystalline forms.

The crystalline forms may be isolated by the techniques known in the art. For example, it may be isolated by filtration by gravity or by suction, centrifugation, decantation, and the like. After isolation, the solid may optionally be washed. The wet solid obtained from (c) may be dried suitably in a tray dryer, vacuum oven, air oven, fluidized bed dryer, spin flash dryer, flash dryer, and the like. The drying may be carried out between about 45° C. and about 85° C., for example, about 70° C., optionally under reduced pressure. The drying may be carried out for any time periods, such as, for example, about 1 to about 25 hours, or longer, to obtain a desired mixture of crystalline Forms A and B of palonosetron hydrochloride.

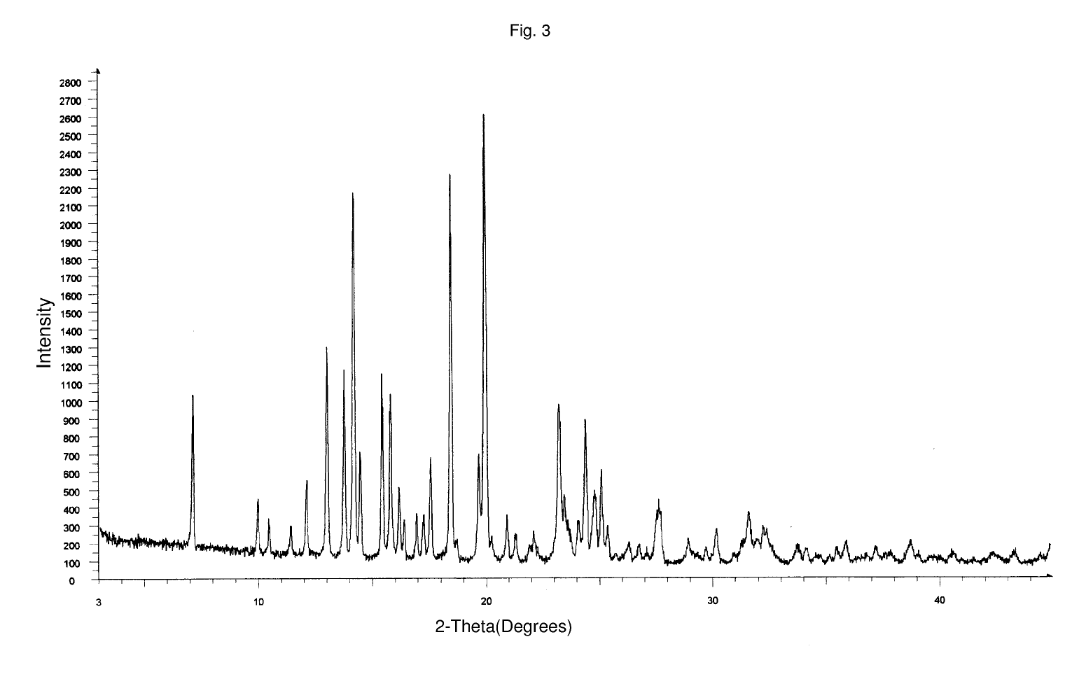

A representative mixture of crystalline Forms A and B of palonosetron hydrochloride obtained by the process of the present invention has an XRPD pattern substantially in accordance with FIG. 3.

Crystalline palonosetron hydrochloride of a defined particle size may be produced by known methods of particle size reduction starting with crystals, powder aggregates, and course powders of the crystalline forms of palonosetron hydrochloride. For example, particle size reduction may be achieved by milling a feedstock material and sorting of the milled particles by size.

The invention includes pharmaceutical compositions comprising a therapeutically effective amount of crystalline palonosetron hydrochloride prepared according to the processes of the present invention, and at least one pharmaceutically acceptable excipient.

Pharmaceutical compositions may be prepared as medicaments to be administered orally, parenterally, transdermally, or nasally. Suitable forms for oral administration include tablets, compressed or coated pills, dragees, sachets, hard or gelatin capsules, sub-lingual tablets, syrups, and suspensions. Suitable forms of parenteral administration include an aqueous or non-aqueous solution or emulsion. For topical administration, the present invention includes suitable transdermal delivery systems known in the art. For nasal delivery, there are provided suitable aerosol delivery systems known in the art. In addition to the active ingredients, the pharmaceutical compositions of the invention contain one or more excipients or adjuvants. Selection of excipients and the amounts to use may be readily determined by the formulation scientist based upon experience and consideration of standard procedures and reference works in the field.

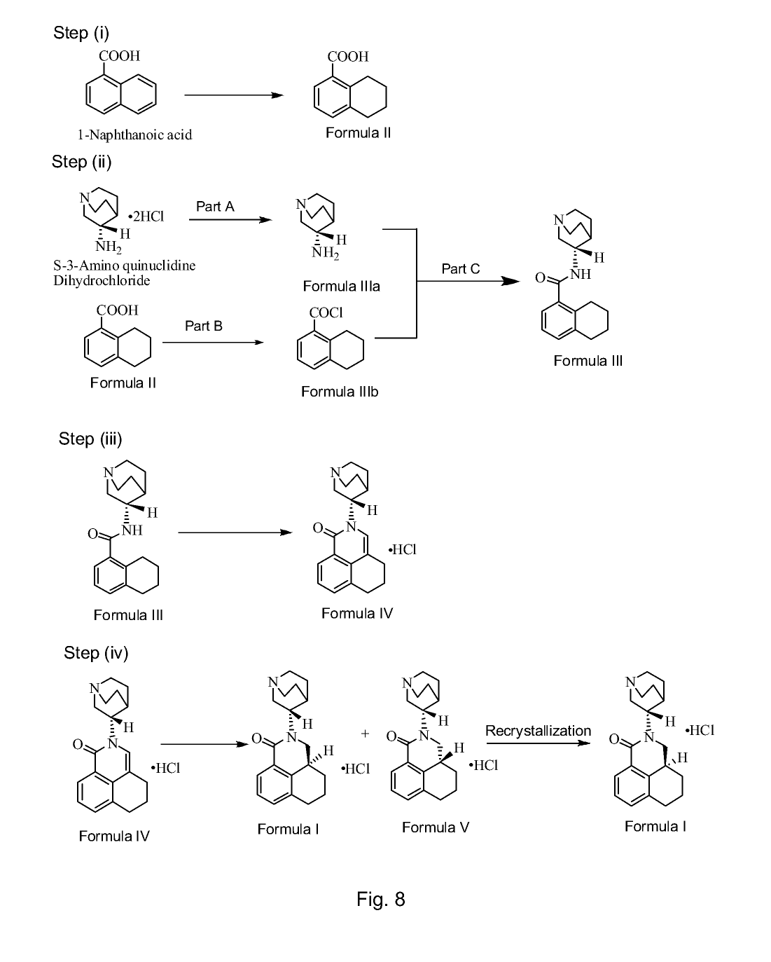

Palonosetron hydrochloride used in the processes of the present invention may be prepared by a process summarized in the scheme of FIG. 8. This process for preparing the palonosetron hydrochloride comprises four steps, called step (i) through step (iv). Step (ii) comprises three sub-steps, enumerated as Part A, Part B and Part C. The specifics of individual steps are discussed hereinbelow.

Step (i) includes reduction of 1-naphthaoic acid using 10% Pd/C in the presence of an alcohol or an organic acid, to provide 5,6,7,8-tetrahydro-naphthalene-1-carboxylic acid of Formula II.

The reduction process may be carried out using about 35% to about 50% by weight wet 10% Pd/C, which can be types 487 or 489 or equivalent grades, in the presence of hydrogen gas. The quantity of Pd/C used for the reduction may range from about 15 to about 30% by weight of the amount of 1-naphthoic acid.

The alcohol solvents that may be used for the reduction include, but are not limited to, n-propanol, isopropanol, or mixtures thereof. For example, n-propanol is used as the solvent.

The organic acids that may be for the reduction include, but are not limited to, acetic acid, formic acid, and the like. For example, acetic acid is used as the solvent.

The reduction may be carried out at temperatures about 40° C. or higher, depending upon the solvent used. For example, the reduction may be carried out at temperatures about 70° C. to about 90° C.

After completion of the reaction, the reaction mixture may be filtered to remove Pd/C under hot or cooled conditions and the solid washed with an alcohol or an acid, followed by precipitation of the product compound from the obtained filtrate.

Alternatively, when the reaction is carried out using an alcohol solvent, the reaction mixture may be filtered to remove Pd/C under hot or cooled conditions, distilled to a minimum volume and co-distilled with an organic acid, for example acetic acid. The crude product so obtained may be further dissolved in acetic acid and water at temperatures about 50-100° C., followed by cooling to temperatures below 40° C. to precipitate the compound of formula II. The product obtained may optionally be slurried in water at temperatures about 80-85° C.

The compound of Formula II obtained from the present process may have purities greater than about 99% by weight, as determined using HPLC.

Step (ii) involves preparation of the compound of formula III.

PART A: Preparation of S-(−)3-amino quinuclidine free base of formula IIIc.

S-(−)3-amino quinuclidine freebase of formula IIIa may be prepared from S-(−)-3-amino quinuclidine dihydrochloride by reacting with a base such as potassium hydroxide, sodium hydroxide, and the like, in the presence of an alcohol, for example, methanol, ethanol, n-propanol, isopropanol, and the like, at temperatures about 20° C. to the reflux temperature of the solvent used. In embodiments, the reaction may be carried out at temperatures about 25° C. to about 35° C.

Optionally, after completion of the reaction, the alcohol solvent may be replaced with another solvent, for example, a hydrocarbon solvent like toluene, or the solvent used in PART B or PART C of step (ii).

For example, the alcohol may be replaced by distillation under vacuum, optionally by co-distillation with a hydrocarbon such as toluene. The compound of formula IIIa may be isolated, or carried forward in situ to the next reaction.

PART B: Preparation of the compound of formula IIIb by reacting the compound of formula II with thionyl chloride, in the presence of a hydrocarbon.

The amounts of thionyl chloride used may range from about 1 to about 2 molar equivalents, per molar equivalent of the compound of formula II.

The hydrocarbon carbon solvents that may be used include, but are not limited to, toluene.

The reaction may be carried out at temperatures about 20 to about 60° C., or higher.

After completion of the reaction, the reaction mixture may be concentrated to a desired extent and carried forward in situ to the next reaction, or the product may be isolated as a solid using any techniques.

PART C: Preparation of the compound of formula III.

The compound of formula III may be prepared by condensation of the compound 5,6,7,8-tetrahydro-naphthalene-1-carboxylic acid chloride (formula IIIb) obtained in part B) with S-(−)3-amino quinuclidine free base (formula IIIa) obtained in part A), in the presence of a hydrocarbon solvent and a base.

The condensation reaction may be carried out at temperatures about 25° C. to about 70° C., and optionally under an inert, such as a nitrogen, atmosphere. In embodiments, temperatures of 40° C. to 60° C. are employed.

Suitable bases that may be used in the condensation reaction include triethylamine, diisopropylethylamine, and the like.

The hydrocarbon solvents that can be used in the condensation step include, but are not limited to, toluene.

After completion of the reaction, the reaction mixture may be quenched with water to obtain a biphasic medium and treated with base to adjust the mass pH to about 9.5-12. For example, an aqueous solution of sodium hydroxide, potassium hydroxide, sodium carbonate, or potassium carbonate may be used for adjusting the pH. The organic layer may be separated, concentrated to a minimum volume and cooled to temperatures less than about 35° C. to isolate the compound of formula III, which may be optionally recrystallized from a hydrocarbon solvent.

Step (iii) involves cyclization of the compound of Formula III using n-butyl lithium and dimethylformamide, to provide 2-[(S)-1-Azabicylco[2.2.2]oct-3-yl]-2,4,5,6-tetrahydro-1H-benz [de] isoquinolin-1-one hydrochloride of formula IV.

The cyclization reaction may be carried out using about 3 to about 4 molar equivalents of n-butyl lithium, and about 3.5 to 4.5 molar equivalents of dimethylformamide, per molar equivalent of the compound of formula III.

The solvents that may be used include ethers such as dimethyl ether, diethyl ether, diisopropyl ether, tetrahydrofuran (THF), and the like.

The cyclization reaction may be performed at temperatures ranging from about −45° C. to about 0° C.

After completion of the reaction, concentrated hydrochloric acid (HCl), or hydrogen chloride in isopropyl alcohol (IPA HCl), may be added to the reaction mass at the reaction temperature and maintained at room temperature, followed by addition of water and separating the layers. The aqueous layer may be treated with base to adjust the mass pH to about 9-10 and extracted with an organic solvent such as ethyl acetate, dichloromethane, chloroform, and the like. Suitable bases that may be used for adjusting the pH include sodium hydroxide, potassium hydroxide, sodium carbonate, potassium carbonate, and the like. The base may be used in the form of a solid or aqueous solution.

The obtained extracts in the organic solvent may be concentrated under vacuum, optionally co-distilled with an alcohol, for example, isopropanol, and precipitated from the alcoholic solution by adding conc. hydrochloric acid (HCl), or hydrogen chloride in isopropyl alcohol (IPA HCl). The compound of formula IV obtained may be optionally recrystallized from an alcohol, for example isopropanol.

Step (iv) involves reduction of the compound of formula IV using 10% Pd/C in the presence of an alcohol, to provide palonosetron hydrochloride of formula I.

The reduction process may be carried out using wet 10% Pd/C, such as types 487 or 489, or an equivalent grade, in the presence of hydrogen gas. The quantity of Pd/C used for the reduction may range from about 40 to about 60% by weight of the amount of the compound of formula IV.

The alcohol solvents that may be used for the reduction include, but are not limited to, methanol, ethanol, n-propanol, isopropanol, and any mixtures thereof. For example, methanol or n-propanol may be used as the solvent.

The reduction may be carried out at temperatures about 40° C., or higher, depending upon the solvent used. For example, the reduction may be carried out at temperatures about 55° C. to about 60° C.

After completion of the reaction, the mass may be filtered to remove Pd/C under hot or cooled conditions and the solid washed with alcohol or an acid, followed by isolation of the palonosetron HCl.
Palonosetron hydrochloride obtained from (iv) may be directly utilized for making polymorphic Forms A and B.

The above processes for the preparation of palonosetron hydrochloride can prepare pure palonosetron hydrochloride. A crystallization process can optionally be repeated to get substantially pure palonosetron hydrochloride having purity greater than or equal to about 99.9% by weight, as determined using HPLC.

The present invention includes “substantially pure” palonosetron hydrochloride, wherein the amount of each individual process related impurity listed in Table 1 is less than about 0.15%, or less than about 0.1%, or less than about 0.05%, by weight, and/or the sum of all of these impurities is less than about 0.2%, by weight. Further, palonosetron hydrochloride obtained by a process of the present invention has (3aS, 3R) and (3aR, 3R) isomers below their limits of detection.

TABLE 1

| Impurity | Structure |
|---|---|
| A | 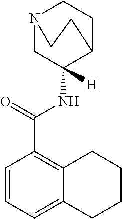 |
| B | 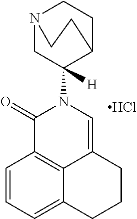 |
| C | 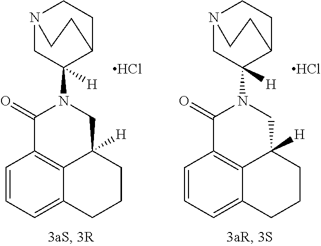 |
| D | 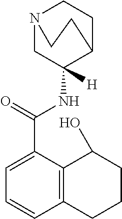 |
| E | 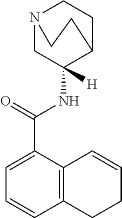 |
| F | 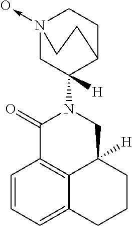 |
| G | 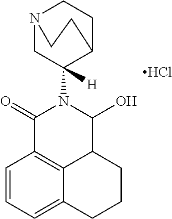 |

In embodiments, the present invention provides compositions comprising palonosetron hydrochloride that contains less than about 0.1% by weight of any individual impurities having structural formulae A, B, C, D, E, F, or G.

The impurities may be analyzed using various methods. Representative useful high performance liquid chromatography (HPLC) methods are described below.

Method 1. Palonosetron hydrochloride may be analyzed by HPLC utilizing the following conditions:

Column: Cosmosil PYE NAP (250×4.6 mm, 5 μm).

Column temperature: 35° C.

Injection volume: 10 μL.

Elution: Gradient.

Concentration: 0.5 mg/mL.

Diluent: Aceonitrile:water (1:1 v/v).

Buffer: Dissolve 3.48 g of K₂HPO₄ and 2 ml of triethylamine in 1000 mL of water and adjust the pH to 2.5 with orthophosphoric acid.

Mobile Phase A: Buffer.

Mobile Phase B: Degassed mixture of buffer and acetonitrile in the volume ratio of 50:50.

Flow rate: 1.0 mL/minute.

Wavelength of detection: 210 nm UV.

Gradient program:

| Minutes | % Mobile Phase B |
|---|---|
| 0 | 40 |
| 45 | 85 |
| 55 | 95 |
| 60 | 100 |
| 63 | 40 |
| 70 | 40 |

Method 2. Palonosetron hydrochloride may also be analyzed by HPLC utilizing the following conditions:

Column: Chiral CEL-OD-H (250 mm×4.6 mm, 5 μm).

Column temperature: 25° C.

Injection volume: 20 μL.

Diluent: Mobile phase.

Flow rate: 1.0 mL/minute.

Wavelength of detection: 240 nm UV.

Mobile Phase: a mixture of n-hexane, ethanol, methanol, diethylamine, and trifluoroacetic acid (900:50:50:2:0.5 by volume).

X-ray powder diffraction patterns described herein are generated using copper Kα radiation with a Rigaku Dmax 2200 instrument equipped with a RINT2000 wide-angle goniometer having a scintillation counter detector. Patterns are recorded at a tube voltage of 50 kV and a tube current of 34 mA with a step size of 0.02° and time per step of 3°/minute over an angular range of 3-45 degrees 2-theta.

Differential scanning calorimetry (DSC) curves are generated using a Q1000 model instrument from TA Instruments, New Castle, Del. USA, with a 10° C./minute heating rate.

Thermogravimetric analysis (TGA) curves are generated using a Q500 model instrument from TA Instruments, with a 10° C./minute heating rate.

Certain specific aspects and embodiments will be further described in the following examples, which are provided only for purposes of illustration and are not to be construed as limiting the scope of the invention. In the examples, percentages are expressed on a weight basis, unless the context indicates otherwise.

### Example 1

### Preparation of Palonosetron Hydrochloride

Step (i): Preparation of 5,6,7,8-tetrahydro-1-naphthalene carboxylic acid (formula II).

1-Naphthoic acid (50 g) and acetic acid (300 mL) are charged into a hydrogenation flask, and 10% Pd/C (50% wet; 10 g) is added. The reaction vessel is flushed twice with hydrogen gas. Hydrogen pressure of 4-5 Kg/cm² is applied, and the mass is heated to 80-85° C. The mass is maintained at this temperature and pressure until completion of the reaction. After completion of the reaction, stirring is stopped, the catalyst is allowed to settle, and the hydrogen pressure is released. The mass is filtered at 80-85° C. and washed with acetic acid (100 mL). The filtrate is charged into a round bottom flask and water (400 mL) is added slowly at 35° C. over about 30-60 minutes. The mass is stirred at 25-35° C. for 30-60 minutes, filtered, the solid washed with water (2×100 mL) and the product obtained is dried at 70-75° C. Yield: 38 g (74.3%). Purity by HPLC: 99.54%.

Step (ii): Preparation of N—[(S)-1-Azabicylco[2.2.2]oct-3-yl]-5,6,7,8-tetrahydro-1-naphthalenecarboxamide (formula III).

Part A: Preparation of S-(−)3-amino quinuclidine free base (Formula IIIa).

Methanol (600 mL) and potassium hydroxide (89 g) are charged into a round bottom flask and stirred. S-(−)-3-amino quinuclidine dihydrochloride (135.7 g) is added and the mass is heated to reflux temperature (−62° C.). The mass is stirred at reflux temperature for 2 hours. The mass is concentrated at 60-65° C. using vacuum until the solvent has been distilled. Toluene (200 mL) is charged to the residue and the distillation is continued until no more solvent distills. Toluene (800 mL) is charged to the residue, and the mixture is heated to 60-65° C. and maintained for 20-30 minutes. The mass is filtered and the filtrate is washed with toluene (500 mL). The filtrate is charged into a round bottom flask and residual water is removed azeotropically at reflux temperature until no more water is collected, after which the mass is cooled to 40-50° C.

Part B: Preparation of 5,6,7,8-tetrahydro-1-naphthalene carboxylic acid chloride (formula IIIb).

5,6,7,8-Tetrahydronapthoic acid (100 g) and toluene (500 mL) are charged into a round bottom flask at 28° C. and stirred. Dimethylformamide (0.6 mL) is added and then thionyl chloride (50 mL) is added drop-wise at 25-35° C. over 15-30 minutes. The temperature is raised to 40-45° C. and the mass is stirred for 1-2 hours. The mass is concentrated at temperatures below 60° C. under vacuum, until no more solvent is distilled, then toluene (200 mL) is added. The mass is concentrated below 60° C. under vacuum, until no more solvent distills. The obtained mass is dissolved in toluene (500 mL).

Part C: Preparation of N—[(S)-1-Azabicylco[2.2.2]oct-3-yl]-5,6,7,8-tetrahydro-1-naphthalenecarboxamide (formula III).

The acid chloride solution of Part B is combined with the free amine of Part A under a nitrogen atmosphere at 40-60° C. The mass is heated to 60-65° C. and maintained at that temperature for 1-2 hours. Water (300 mL) is added at 55-60° C. The mass is made basic by adding 10% NaOH solution (500 mL). The mass is stirred for 10-20 minutes at 55-60° C. and the organic layer is separated. The aqueous layer is extracted with toluene (300 mL) at 55-60° C. and the organic layers are combined and washed with water (300 mL). The organic layer is concentrated under vacuum at 50-55° C. until no more solvent distills. Toluene (600 mL) is charged to the residue. The mass is cooled to 10-15° C. and maintained at that temperature for 30-60 minutes. The mass is filtered, washed with toluene (200 mL), and dried at 65-70° C.

Yield: 146.0 g (88.6%).

Purity by HPLC: 98.22%.

Step (iii): Preparation of 2-[(S)-1-Azabicylco[2.2.2]oct-3-yl]-2,4,5,6-tetrahydro-1H-benz [de] isoquinolin-1-one hydrochloride (Formula IV).

N—[(S)-1-Azabicylco[2.2.2]oct-3-yl]-5,6,7,8-tetrahydro-1-naphthalene carboxamide (100 g) and tetrahydrofuran (1200 mL) are charged into a round bottom flask at 28° C. under a nitrogen atmosphere and stirred at 25-35° C. for 10-15 minutes. The mass is cooled to −33° C. and n-butyl lithium (1.6 M in hexane; 773.3 mL) is added at −35 to −25° C. over 30-60 minutes. The mass is stirred for 10-20 minutes. Dimethylformamide (108.9 mL) is added drop-wise at −35 to −25° C. over 15-30 minutes and the mass is stirred for 30-60 minutes at the same temperature. Conc. HCl (36%; 254.4 mL) is added at −35 to 0° C. over 30-60 minutes. The pH of the mass is less than 2. The temperature of the mass is raised to 25-35° C. The mass is stirred for 1-2 hours at 25-35° C., water (500 mL) is added, and the mixture is stirred for 10-15 minutes. The layers are separated, the aqueous layer is placed into a round bottom flask, and 50% NaOH is added to the aqueous layer at 25-35° C. to produce a pH of 11-12. The mass is stirred for 10-15 minutes and extracted with ethyl acetate (500 mL+200 mL), then the combined organic layers are washed with water (300 mL). The organic layer is concentrated below 63° C. under vacuum until no more solvent distills. The residue is co-distilled with isopropyl alcohol (50 mL) below 63° C. under vacuum. Isopropyl alcohol (200 mL) is added to the residue, heated to 50-55° C. for dissolution, and the solution is cooled to 25-35° C. Hydrogen chloride in isopropyl alcohol (18%; 165 mL) is added drop-wise over 10-15 minutes at 25-35° C. and maintained for 1-2 hours. The solid is filtered under a nitrogen atmosphere, washed with isopropyl alcohol (100 mL), and dried at 65-70° C.

Yield: 95.0 g (79%).

Purity by HPLC: 98.95%.

Step (iv): Preparation of 3aS-[2-[(S)-1-Azabicylco[2.2.2]oct-3-yl]-2,3,3a,4,5,6-hexahydro-1H-benz[de]isoquinolin-1-one hydrochloride (formula I).

2-[(S)-1-azabicylco[2.2.2]oct-3-yl]-2,4,5,6-tetrahydro-1H-benz[de] isoquinolin-1-one hydrochloride (50 g), 10% Pd—C (50% wet; 50 g), and methanol (500 mL) are charged into a hydrogenation flask at 28° C. The vessel is flushed twice with hydrogen gas. The mass is stirred for 5 minutes and a hydrogen pressure of 10-11 Kg/cm² is applied at 25-35° C. The temperature is raised to 55-60° C. under 10-11 Kg/cm² hydrogen pressure and maintained until completion of the reaction. The mass is cooled and the pressure is released. The mass is filtered, the solid is washed with methanol (100 mL), and the filtrate is concentrated at 55-60° C. under vacuum to give 48.5 g (wet) of crude palonosetron hydrochloride.

Purity by HPLC: 54.60% (palonosetron hydrochloride).
43.65% (3aR isomer of Formula V).

XRPD pattern: as in FIG. 3.

Acetone (240 mL) is combined with 24.3 g of the obtained crude wet palonosetron hydrochloride, heated to reflux, and maintained for 2 hours. The mass is filtered and the solid is washed with acetone.

Purity by HPLC: 87.49% (palonosetron hydrochloride).
12.10% (3aR isomer-Formula V).

XRPD pattern: as in FIG. 2.

The wet product and acetone (180 mL) are charged into a round bottom flask, heated to reflux temperature, and maintained for 2 hours. The hot mass is filtered and the solid is washed with acetone (40 mL).

Purity by HPLC: 88.95% (palonosetron hydrochloride).
10.78% (3aR isomer-Formula V).

XRPD pattern: substantially in accordance with FIG. 2.

The wet product and acetone (150 mL) are charged into a round bottom flask, heated to reflux temperature, and maintained for 2 hours. The hot mass is filtered and the solid is washed with acetone (30 mL) and dried at 71° C. to give 10.4 g of crystalline palonosetron hydrochloride. Yield: 10.4 g.

Purity by HPLC: 91.41% (palonosetron hydrochloride).
8.33% (3aR isomer-Formula V).

XRPD pattern: as in FIG. 2.

The crystalline palonosetron (10.4 g) obtained above and n-propanol (70 mL) are charged into a round bottom flask, heated to reflux temperature, and maintained for 2 to 3 hours. The hot mass is filtered and the solid is washed with n-propanol (5 mL) and dried at 71° C. to give 4.4 g of crystalline palonosetron hydrochloride.

Purity by HPLC: 99.88% (palonosetron hydrochloride).
3aR isomer (Formula V)—not detected.

XRPD pattern: as in FIG. 1.

### Example 2

### Preparation of Crystalline Form A Using Acetonitrile

Palonosetron hydrochloride (1 g) and acetonitrile (15 mL) are charged into a round bottom glass flask and stirred. The mixture is heated to reflux (˜80° C.) and maintained at this temperature for about 2 hours. The hot suspension is filtered and the solid is washed with acetonitrile (10 mL). The solid is dried at 71° C. to obtain palonosetron hydrochloride, with a 65% yield.

XRPD pattern: as in FIG. 1.

### Example 3

### Preparation of Crystalline Form A Using N-Propyl Alcohol

Palonosetron hydrochloride (1 g) and n-propyl alcohol (7 mL) are charged into a round bottom glass flask and stirred. The mixture is heated to reflux temperature (˜89° C.) and maintained at this temperature for about 2 hours. The hot suspension is filtered and the solid is washed with n-propyl alcohol (3 mL). The solid is dried at 71° C. to obtain palonosetron hydrochloride, with a 60% yield.

XRPD pattern: as in FIG. 1.

### Example 4

### Preparation of Crystalline Form B Using Acetone

Palonosetron hydrochloride (50 g), 10% Pd—C (50% wet; 50 g), and methanol (500 mL) are charged into a hydrogenation flask at 28° C., and the vessel is flushed twice with hydrogen gas. The mass is stirred for 5 minutes and a hydrogen pressure of 10-11 Kg/cm² is applied at 25-35° C. The mass temperature is raised to 55-60° C. under 10-11 Kg/cm² hydrogen pressure and maintained until completion of the reaction. The mass is cooled and the pressure is released. The mass is filtered, washed with methanol (100 mL), and concentrated at 55-60° C. under vacuum. The obtained residue is co-distilled with acetone (2×200 mL). Acetone (500 mL) is added to the residue, heated to reflux and maintained for 1-2 hours. The mass is filtered and the solid is washed with hot acetone (100 mL) and dried at 70° C. to obtain palonosetron hydrochloride.

XRPD pattern: as in FIG. 2.

### Example 5

### Preparation of Crystalline Form a Using N-Propyl Alcohol

Palonosetron hydrochloride Form B (24 g) obtained as in Example 4 and n-propyl alcohol (168 mL) are charged into a round bottom glass flask and stirred. The mixture is heated to reflux temperature and maintained at that temperature for about 2.5 hours. The hot suspension is filtered and the solid is washed with n-propyl alcohol (12 mL). The solid is dried at 71° C. to obtain palonosetron hydrochloride with a 52% yield.

XRPD pattern: as in FIG. 1.

### Example 6

### Preparation of a Mixture of Crystalline Forms A and B Using acetonitrile

Palonosetron hydrochloride (1 g) and acetonitrile (125 mL) are charged into a round bottom glass flask and stirred. The mixture is heated to reflux temperature and maintained at that temperature for about 4 hours. The mass is cooled to 28° C. and maintained at that temperature for 2.25 hours. The suspension is filtered and the solid is washed with acetonitrile (10 mL). The solid is dried at 71° C. to obtain palonosetron hydrochloride with a yield of 80%.

XRPD pattern: as in FIG. 3.

### Example 7

### Preparation of a Mixture of Crystalline Forms A and B Using Acetonitrile and Methanol

Palonosetron hydrochloride (1 g), acetonitrile (18.8 mL), and methanol (0.2 mL) are charged into a round bottom glass flask and stirred. The mixture is heated to reflux temperature (˜79° C.) and maintained at that temperature for about 2 hours. The mass is cooled to 30° C. and maintained at that temperature for 1 hour. The suspension is filtered and the solid is washed with acetonitrile (10 mL). The solid is dried at 71° C. to obtain palonosetron hydrochloride with a yield of 58%.

XRPD pattern: as in FIG. 3.

### Example 8

### Preparation of a Mixture of Crystalline Forms A and B Using N-Propyl Alcohol

Palonosetron hydrochloride (1 g) and n-propyl alcohol (30 mL) are charged into a round bottom glass flask and stirred. The mass is heated to reflux temperature (˜90° C.) to obtain a clear solution and maintained at that temperature for about 50 minutes. The mass is cooled to 29° C. and maintained at that temperature for 1 hour. The suspension is filtered and the solid is washed with n-propyl alcohol (5 mL). The solid is dried at 71° C. to obtain palonosetron hydrochloride with a yield of 50%.

XRPD pattern: as in FIG. 3.

### Example 9

### Preparation of 3aS-[2-[(S)-1-Azabicylco[2.2.2]oct-3-yl]-2,3,3a,4,5,6-hexahydro-1H-benz[de]isoquinolin-1-one hydrochloride (Formula I)

2-[(S)-1-azabicylco[2.2.2]oct-3-yl]-2,4,5,6-tetrahydro-1H-benz[de] isoquinolin-1-one hydrochloride (100 g), 10% Pd—C (50% wet; 100 g), and n-propanol (1000 mL) are charged into a hydrogenation flask at 30° C. The vessel is flushed twice with hydrogen gas (4-5 Kg/cm²). The mass is stirred for 20 minutes and a hydrogen pressure of 10-11 Kg/cm² is applied at 25-35° C. The temperature of the mass is raised to 55-60° C. under 10-11 Kg/cm² hydrogen pressure and maintained until completion of the reaction (20 hours). The mass is cooled and the pressure is released, the mass is filtered and the solid is washed with n-propanol (200 mL). The filtrate is concentrated at 95-100° C. under vacuum to a volume of 300-400 mL, refluxed for 3-4 hours and cooled to 25-30° C. over 1 hour. The suspension is filtered and the solid is washed with n-propanol (200 mL) and suction dried for 30 minutes under a nitrogen atmosphere.

The wet compound (40 g) and methanol (120 mL) are charged into a round bottom flask at 25-30° C. The mixture is heated to reflux and stirred for 3 hours. The mixture is cooled to 0-5° C., stirred at that temperature for 3 hours, filtered, and the solid is washed with methanol (40 mL). The solid is dried at 60-65° C. for 5 hours under vacuum, to afford 32 g of crude palonosetron hydrochloride.

Purity by HPLC: 98.93% (palonosetron hydrochloride); Impurity A: not detected; Impurity B: 0.13%; Impurity C, 0.91%; Impurity D: not detected; Impurity E: not detected; Impurity F: not detected; Impurity G: not detected.

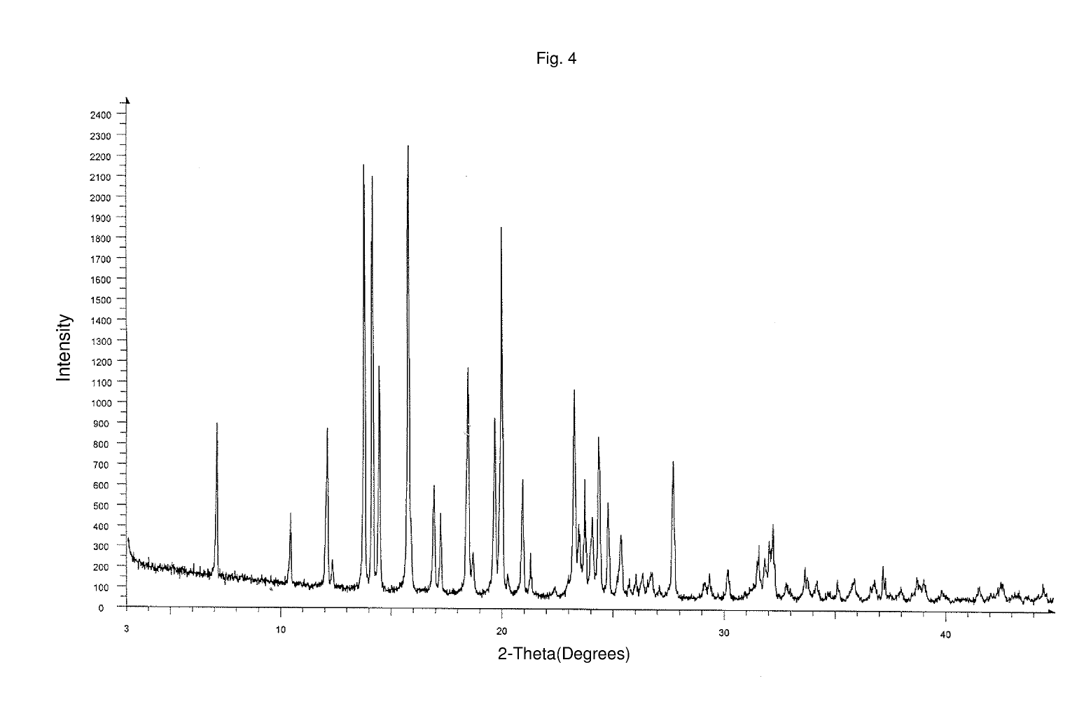

XRPD pattern: as in FIG. 4.

### Example 10

### Purification of Palonosetron Hydrochloride

Palonosetron hydrochloride (40 g, purity: 98.5%, 3aR isomer: 1.1%) is dissolved in methanol (800 mL) at 25-30° C. Acidic carbon (8 g) is charged and stirred for 20 minutes. The mixture is filtered through a Hyflow (flux-calcined diatomaceous earth) bed and the bed is washed with methanol (80 mL). The filtrate is distilled at 65-70° C. to afford a suspension (approximately 120-160 mL of methanol being present in the suspension) and refluxed at that temperature, followed by stirring for 3 hours. The suspension is cooled to 0-5° C., stirred for 1 hour and filtered. The solid is washed with n-propanol (200 mL), followed by chilled methanol (40 mL), and suction dried.

The obtained wet cake is suspended in methanol (85 mL), heated to reflux temperature, and stirred for 1-2 hours. The mass is cooled to 0-5° C. and stirred for 2 hours. The suspension is filtered, and the solid is washed with n-propanol (154 mL) followed by chilled methanol (30 mL), and suction dried.

The obtained wet cake is suspended in methanol (60 mL), heated to reflux temperature and stirred for 1-2 hours. The mass is cooled to 0-5° C. and stirred for 2 hours. The suspension is filtered, and the solid is washed with n-propanol (100 mL) followed by chilled methanol (20 mL) and suction dried. The solid is dried at 70° C. under vacuum for 9 hours to afford 13 g of the title compound.

Purity by HPLC: 99.89% (palonosetron hydrochloride); Impurity A: not detected; Impurity B: 0.04%; Impurity C, 0.05%; Impurity D: not detected; Impurity E: not detected; Impurity F: not detected, Impurity G: not detected.

XRPD pattern: as in FIG. 5.

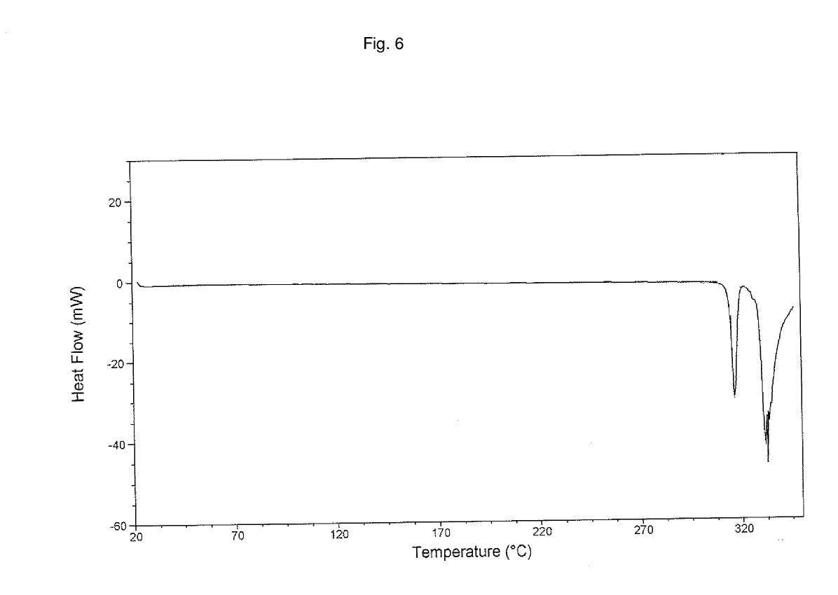

DSC curve: as in FIG. 6.

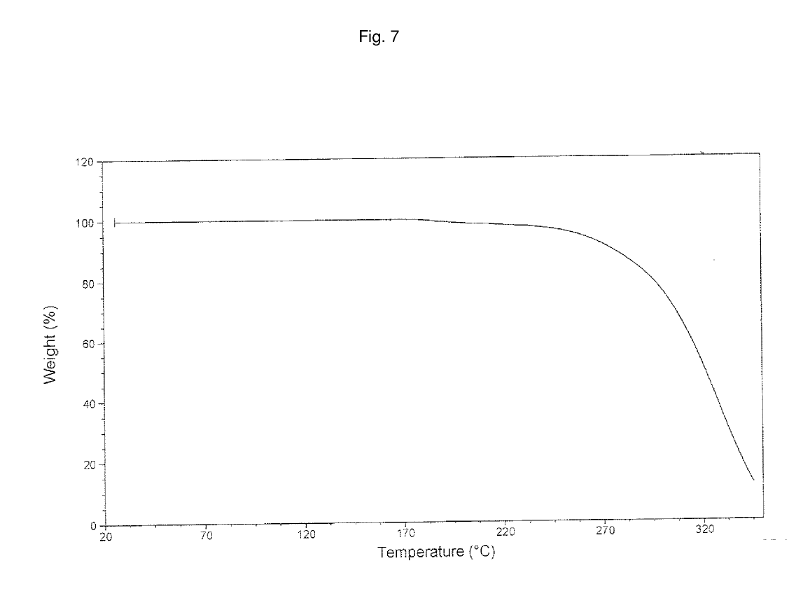

TGA curve: as in FIG. 7.

1. A process for preparing palonosetron hydrochloride, comprising:
(a) hydrogenating a compound of formula IV, 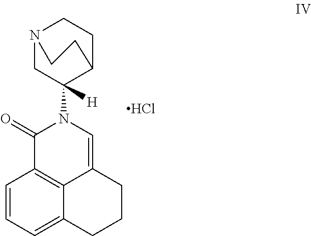
in the presence of a hydrogenation catalyst and n-propanol;
(b) providing a suspension of a product of (a) in methanol;
(c) maintaining the suspension at temperatures about 45° C. to the reflux temperature; and
(d) isolating palonosetron hydrochloride.

2. The process of claim 1, wherein a hydrogenation catalyst comprises palladium on carbon, palladium on barium sulfate, or platinum oxide.

3. The process of claim 1, wherein an amount of hydrogenation catalyst is about 30% to about 60% by weight of the compound of formula IV.

4. The process of claim 1, wherein hydrogenation is carried out at about 55° C. to about 60° C.

5. The process of claim 1, wherein hydrogenation is carried out with hydrogen gas pressures about 8-12 Kg/cm².

6. The process of claim 1, wherein a product from (a) is isolated by concentration or precipitation.

7. The process of claim 1, wherein a suspension in (b) is provided by combining a product of (a) with methanol.

8. The process of claim 1, wherein in (c) the suspension is maintained at reflux temperature.

9. The process of claim 1, further comprising repeating (b) through (d) at least once.

10. A process for preparing crystalline palonosetron hydrochloride crystalline Form A, comprising:
(a) providing a suspension of palonosetron hydrochloride in n-propyl alcohol or a nitrile solvent;
(b) maintaining the suspension at temperatures about 50° C. to the boiling point of the solvent; and
(c) isolating crystalline Form A.

11. The process of claim 10, wherein a nitrile solvent comprises acetonitrile or propionitrile.

12. The process of claim 10, wherein the suspension is maintained at temperatures about 80° C. to about 100° C.

13. A process for preparing palonosetron hydrochloride crystalline Form B, comprising:
(a) providing a suspension of palonosetron hydrochloride in a ketone solvent, optionally in combination with an alcohol;
(b) maintaining the suspension at suitable temperature; and
(c) isolating crystalline Form B.

14. The process of claim 11, wherein a ketone solvent comprises acetone, methyl ethyl ketone, or methyl isobutyl ketone, and an alcohol solvent comprises methanol, ethanol, or isopropanol.

15. The process of claim 11, wherein the suspension is maintained at temperatures about 40° C. to the reflux temperature of the ketone solvent.

16. A process for preparation of a mixture of crystalline Forms A and B of palonosetron hydrochloride, comprising:
(a) providing a suspension or solution of palonosetron hydrochloride in n-propyl alcohol or a nitrile solvent, optionally in combination with a second alcohol or a ketone solvent, or mixtures thereof;
(b) maintaining the suspension or solution at temperatures about 0° C. to about 40° C.; and
(c) isolating a mixture of crystalline Forms A and B.

17. The process of claim 11, wherein a nitrile solvent comprises acetonitrile or propionitrile, a ketone solvent comprises acetone, methyl ethyl ketone, or methyl isobutyl ketone, and an alcohol solvent comprises methanol, ethanol, or isopropanol.

18. A pharmaceutical composition prepared using palonosetron hydrochloride having less than about 0.1% by weight of any individual impurity having structural formula A, B, C, D, E, F, or G. 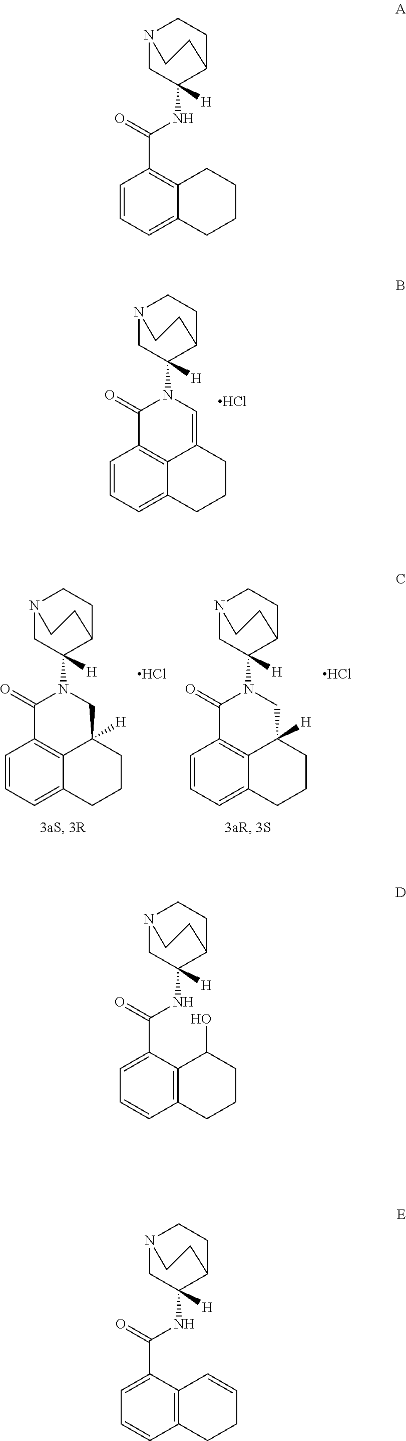 [chemical structure]
